(19) United States
(12) Patent Application Publication
(10) Pub. No.: US 2017/0196904 A1
(43) Pub. Date: Jul. 13, 2017

(54) SILICONE OIL-CONTAINING FORMULATIONS FOR NASAL APPLICATION

(71) Applicant: INQPHARM GROUP SDN BHD, Kuala Lumpur (DE)

(72) Inventors: Nowak GÖTZ, Erfurt (DE); Christof JAENICKE, Berlin (DE)

(21) Appl. No.: 15/315,106

(22) PCT Filed: May 28, 2015
(86) PCT No.: PCT/EP2015/001091

§ 371 Date: Nov. 30, 2016

(30) Foreign Application Priority Data
Jun. 3, 2014 (DE) 10 2014 009 164.3

(51) Int. Cl. A61K 31/80; A61K 47/20; A61K 45/06; A61K 31/4164; A61K 9/00; A61K 9/107

(52) U.S. Cl. CPC A61K 31/80; A61K 9/0043; A61K 9/107; A61K 45/06; A61K 31/4164; A61K 47/20

(57) ABSTRACT

Silicone oil-containing formulation for nasal application for use in a method for intranasal treatment of endogenous diseases of the nose and/or those due to exogenous causes, e.g. those which are caused by bacteria, fungi, viruses and/or allergens, wherein the silicone oil or the formulation is optionally enriched with oxygen, preferably in the presence of emulsifiers, and optionally in the presence of further active ingredients, such as α-sympathomimetic drugs, e.g. oxymetazoline or xylometazoline, and related subjects of the invention.

The invention relates to a silicone oil-containing formulation for nasal application for intranasal treatment of endogenous diseases of the nose, and/or those due to exogenous causes such as bacteria, fungi, viruses or allergens, in particular rhinitis.

Inflammatory diseases of the nasal mucosa are one of the commonest causes of illness and affect several million people of all age groups annually in Germany. Older people and immunodeficient persons especially display severe disease symptoms which are characterized by swelling and oedematization of the nasal mucosae with dyspnoea and complications. Complications feared are accompanying inflammation of the mucosae of the paranasal sinuses due to oedematous mucosal swelling in the direct vicinity of the apertures of these air chambers located in the nasal cavity, which then no longer allow pressure equalization and become purulently inflamed. The commonest causes are rhinoviruses or other viral vectors during the cold season, but influenza viruses can also utilize the nasal mucosal cells as virus generation sites and lead to the typical irritations and secretions. Apart from the viral aetiology, bacterial species also occur in the mucosae of the nose, especially in immuno-deficient persons, which then often lead to chronic nasal inflammation (ozaena) and are difficult to treat. A further common cause of nasal mucosal irritation are allergens, e.g. flower pollen, animal epithelial components and other organic dusts, which can trigger immune reactions on the nasal mucosa and the mucosae of the upper respiratory tract. As a result, severe swelling and irritation are seen simultaneously with liquid nasal secretion. This form of rhinitis is admittedly not infectious in contrast to the viral and bacterial rhinitis forms, but those affected have further general symptoms, such as headache, general exhaustion and malaise. Rhinitis diseases are typical seasonal widespread diseases rarely taking a serious course, but with a high economic impact. Various forms of Rhinitis are thus known: allergic rhinitis (Rhinitis allergica), viral or bacterial or fungally produced (general infectious) Rhinitis, such as for example Rhinitis acuta, further, Rhinitis atrophicans (ozaena), Rhinitis sicca, hypertrophic rhinitis (Rhinitis hypertrophica), Rhinitis vasomotorica, Rhinitis pseudomembranacea, Rhinitis medicamentosa or chronic rhinitis, or two or more thereof. The said diseases also correspond as preferred or specific variants to the term “nasal diseases” or “diseases of the nose” mentioned above and below in the definition of the invention.

For the restoration of the physiological functions of the nose, various local therapeutic principles are applied: aqueous solutions or nasal ointments as active substance carriers are intended to regenerate and moisten the mucosae. As a result, the ciliated epithelial function should be supported and the nose again becomes usable as a respiratory organ. In acute rhinitis, active substance-containing nasal drops, nasal sprays and salt solutions, which restore the openness of the nose for a certain time, are used. Essentially, alpha mimetics are used, which contract the precapillaries of the nasal microcirculation and thus decrease fluid production. However, virostatic agents, and less commonly antibiotics, are also used. Nasal oils, often combined with inflammation-inhibiting plant active ingredients, often decrease mass and gas transfer on the mucosal surface and are intended to create a protective film on the epithelia. With major secretion, these films cannot create the desired therapeutic effect. Here the rhinitis drugs containing vasoconstrictive substances are advantageous, however because of their short duration of action times these locally acting drugs are introduced into the nose too often. On chronic use, the permanently impaired microcirculation leads to capillary vascular insufficiency and microcapillary haemorrhages due to stasis and micro-thrombosis. However, mucosal atrophy and permanent nasal dryness are also sequelae in the chronic use of nasal therapeutics (Rhinitis medicamentosa). Although there is a broad market presence of rhinitis remedies, which are used in the various types of colds, because of the varying aetiology, causal curing or prophylaxis has not so far been possible.

It is known that oxygen-containing silicone oil emulsions can be used for the treatment of gastrointestinal diseases, as described in EP 2 151 242 A1. Similar compositions with no oxygen content are also described in the dissertation by Katja Presselt, “Preparation and characterization of silicone oil emulsions and studies on their therapeutic potential in chronic inflammatory skin diseases” Biologisch-Pharmazeutische Fakultät der Friedrich-Schiller-Universität Jena, 2010.

It has now surprisingly been found that (in particular oxygen-enriched) emulsions of silicone oils in an aqueous base are suitable for the treatment of nasal diseases even without other active ingredients.

Mainly responsible for this is the creep capacity of oil emulsions which due to the surface tension-decreasing action in aqueous solutions can perfectly migrate under mucus deposits of nasal cells and detach them from their substrates. Particularly suitable for this are water-in-oil emulsions of silicone oils as described in more detail below. The curative effect of the dimethicone macromolecules is presumably due to interactions by means of hydrophobic bonds with protein-sugar structures of the broken open paracellular TJ. The sealing of the microlesions caused by dimethicone rapidly leads to a normalization of an otherwise excessive immune response, without impairing or antagonizing this. The simultaneous provision of elemental oxygen on the mucosal surface can contribute to a further increased anti-infective action. Further rhinologically active ingredients can also be incorporated into the dimethicone emulsion, which can result in an additive or cumulative anti-rhinitis effect. As a result, an entirely novel anti-rhinitis principle and system is available, which displays no side-effects whatsoever, is not absorbed and can have an 8-10 hour palliative or curative action. Furthermore, a nasal spray with this (in particular oxygen-enriched) dimethicone emulsion is suitable for prophylactic use. If the nasal/pharyngeal mucosa is wetted with the monopreparation oxygen-dimethicone spray before the start of contact with for example rhinoviruses or allergens, a reliable prophylaxis for several (e.g. 12 or more) hours is ensured. Thus, this prophylactic spray can also be provided for self-protection of immunodeficient persons or those with intensive contact with the public (sales personnel, nurses).

The mechanisms of action stated above and below should not be understood as limiting.

In initial applications of such novel nasal sprays, an oxygen-containing 10% dimethicone emulsion without or in particular with oxymetazoline or xylometazoline was tested in patients with acute rhinitis. Here with the application of the active substance combination a marked additive curative effect was seen, and after a single application nasal breathing was often ensured in patients for up to 10 hours.

After repeated application, an acute rhinitis was cured after for example 3-4 days. This combination was particularly effective in allergic fever (“hay fever”). Within a few hours those affected were symptom-free and with spray application once to twice daily could withstand the temporally limited aerogenic allergen exposure without symptoms of illness. In cell experiments with a laryngeal cell line, an effect directed against bacterial penetration could be found.

In spite of the extreme exposure of the nasal cavity to external noxae such as allergens and pathogens (e.g. viruses and bacteria), a positive effect is thus found. It is also surprising that in spite of the presence of oxygen in the air, a stronger effect can be found with oxygen-containing emulsions than with oxygen-free emulsions. Without this explanation being limiting, the effect could be attributable to a masking action, which also helps against viruses.

Thus even against the background of the effects in EP 2 151 242 A1, which only related to the gastrointestinal tract with much lower exposure to pathogens (the nasal cavity acts here as a kind of prefilter), there was a surprising activity.

In a first embodiment, the invention therefore relates to a silicone oil-containing formulation for nasal use, in particular an emulsion of silicone oil in aqueous medium, for the intranasal treatment of endogenous diseases of the nose and/or those due to exogenous causes, such as bacteria, fungi, viruses or allergens, in particular corresponding diseases of the nasal mucosa, in a mammal, in particular human, wherein the silicone oil is preferably enriched with oxygen.

A further embodiment of the invention relates to the use of a silicone oil-containing formulation for nasal use, in particular an emulsion of silicone oil in aqueous medium, for producing a pharmaceutical formulation for intranasal use for the treatment of endogenous diseases of the nose and/or those due to exogenous causes, such as bacteria, fungi, viruses or allergens, in particular corresponding diseases of the nasal mucosa, in a mammal, in particular human, wherein the silicone oil is preferably specifically enriched with oxygen.

A further embodiment of the invention relates to a process or a method for the intranasal treatment of endogenous diseases of the nose and/or those due to exogenous causes, such as bacteria, fungi, viruses or allergens, in particular corresponding diseases of the nasal mucosa, comprising the nasal administration of a silicone oil-containing formulation for nasal use, in particular an emulsion of silicone oil in aqueous medium, to a mammal, in particular a human, in particular which requires such a treatment, in a quantity effective against the said diseases of the nose, wherein the silicone oil is preferably specifically enriched with oxygen.

The following definitions make it possible to replace more general terms above and below by more specific terms, wherein in the embodiments of the invention one or more or all of the general terms can be replaced by specific terms, which leads to more specific, preferred embodiments of the invention.

An emulsion of silicone oil in aqueous medium is in particular an oil-in-water (O/W) emulsion of polydialkyl- or polydiphenylsilanes liquid at room temperature (here defined as 23° C.), in particular of polydi-(C₁-C₇-alkyl)-silanes, in particular of polydimethylsiloxanes (dimethicones) liquid at room temperature.

Examples are polydimethylsiloxanes of the formula I,

(H₃C)₃—Si—O[—Si(CH₃)₂—O]ₙ—Si(CH₃)₃ (I)

with (on average, based on the number of molecules) n=1 to 1000 (the formula serves mainly for illustration), preferably n=1 to 500.

Preferred are siloxanes with average kinematic viscosities (according to manufacturers' data) of 10 to 5000 mm² s⁻¹ at 25° C., for example dimethicones with the designations M10, M50, M100, M350, M500, M1000, M2000 and M5000, in particular M10, M50, M100, M350 and M500, especially M1000 and M2000, (GE Bayer Silicones GmbH & Co KG, Leverkusen, Germany; Wacker Chemie GmbH, Munich, Germany; Caesar & Loretz GmbH, Hilden, Germany), where the numbers in each case state the kinematic viscosities in mm² s⁻¹ at 25° C., which can be measured as follows:

The viscosity of the aqueous solutions can for example be determined with the automatic measuring instrument AVS 3650 (Schott-Geräte GmbH, D-Hofheim), which is combined with the temperature control unit CT 1450/CK 101. For the measurement of the samples at 20° C. and 25° C., various Ubbelohde capillary viscometers are used (Tab. 3-9).

Tab. 3-9: Summary of the Ubbelohde capillary viscometers used (capillary type, classification according to Ph. Eur., instrument number, capillary constants) capillary type according to Ph. Eur.
Instr. Number Constants [mm²/s⁻¹]:
53200 0 97953 0.001038
53201 0a 91414 0.004872
53213 Ic 96276 0.02933
53213 Ic 96277 0.2910
53710 I 1008446 0.009970

The capillary viscometers are rinsed with ethanol before each measurement and then dried with compressed air. The kinematic viscosity of the samples is calculated from the measured passage time of a defined sample volume through the capillary. The measurements are assessed using the software VPC 38. 3-5 measurements are performed for each sample.

Particularly preferred is dimethicone with a kinematic viscosity in the range from 100 to 10,000, in particular from 500 to 5000, e.g. from 1000 to 2000 mm² s⁻¹.

The content by weight of the siloxanes in the emulsions used according to the invention is about 0.1 to 50 wt. %, e.g. 0.5 to 50 wt. %, e.g. 5 to 25 wt. %.

In order to stabilize the emulsion, emulsifiers are necessary.

As emulsifiers, ionogenic emulsifiers (such as sodium dodecylsulphate) or preferably nonionogenic emulsifiers, e.g. low molecular weight nonionogenic emulsifiers or block copolymers, such as

Macrogol glycerol ricinoleates, such as Cremophor® EL (Caesar & Loretz GmbH, D-Hilden);

Polyoxyethylene sorbitan fatty acid esters, such as Tween® 80 (Caesar & Lorentz, D-Hilden);

Mixtures of partial fatty acid esters of sorbitol and anhydrides thereof with oleic acid, e.g. Span® 80 (Sigma-Aldrich Chemie GmbH, D-Steinheim);

Phospholipids such as Lipoid S100 (Lipoid GmbH, D-Ludwigshafen) (mixture with phosphatidylcholine, phosphatidylethanolamine, N-acyl-phosphatidylethanolamine, phosphatidylinositol, lysophosphatidylcholine, triglycerides, free fatty acids and DL-alpha-tocopherol);

Block copolymers of ethylene oxide and propylene oxide units, e.g. Poloxamer 188 (e.g. Lutrol®, BASF, D-Ludwigshafen) Hydroxypropylmethylcellulose (e.g. Metolose® from Shin-Etsu Chemical Co., Ltd, J-Tokyo);

Silicone surfactants in the form of copolymers of polydimethylsilanes and organic glycols, such as Belsil® (Wacker Chemie GmbH, D-Munich) or a form thereof obtained by methanol fractionation (designated as HSY 115);

Sucrose esters, such as Ryoto® Sugar Ester (Mitsubishi-Kagaku Foods Co., J-Tokyo), e.g. esters with lauric acid (L-595), esters with myristic acid (M-1695), esters with oleic acid (O-170 or O-1570), or esters with stearic acid (S-570 or S-1570) as the predominant acyl content;

or mixtures of two or more thereof, e.g. of Tween® 80 and Span® 80 (mixture designated as HLB 8.5),

or also particulate solids, e.g. modified silicon dioxide particles, are possible.

Particularly preferred are block copolymers of ethylene oxide and propylene oxide units, such as Poloxamer 188, or, further, sucrose esters with stearic acid as the predominant acyl content, such as S-570, polyoxyethylene sorbitan fatty acid esters, such as Tween® 80, alone or mixed with mixtures of partial fatty acid esters of sorbitol and anhydrides thereof with oleic acid, e.g. Span® 80, macrogol glycerol ricinoleates, such as Cremophor® EL or phospholipids such as Lipoid 5100, or mixtures of two or more thereof.

The content by weight of the emulsifiers, based on the finished emulsions, preferably lies in a range from 0.01 to 20 wt. %, in particular from 1 to 10 wt. %.

As the aqueous medium, pure water, buffers (such as phosphate buffers, pH adjusted with bases such as NaOH or with phosphoric acid) or other salt solutions (e.g. hypotonic, isotonic or hypertonic solutions of sodium chloride or of potassium chloride) dissolved in water can be used, or also seawater. The content of the aqueous medium in the whole composition preferably lies at 40 to 99.9 wt. %, for example at 60 to 99 wt. %.

Further possible additives are selected from thickeners (“quasi-emulsifiers”) e.g. gum Arabic, gum tragacanth; polyalkylene glycols such as polyethylene glycol or polypropylene glycol, sugar alcohols or other substances which can serve as regulators of the osmotic activity, such as sorbitol, hexitol or mannitol, (in particular in the presence of active ingredients and/or in the absence of oxygen) preservatives such as ethylenediaminetetraacetic acid, antioxidants such as ascorbic acid, BHA or BHT, paraben, benzoic acid, cetyltrimethylammonium chloride, sorbic acid or thiomersal; pigments or dyes, such as beta-carotene, erythrosine, Sunset Yellow FCF, indigotin or the like, aromatizing substances, such as fruit esters or vanillin, and liquid polyols, such as glycerine, propylene glycol or ethylene glycol, can be added preferably in quantities of overall 0 to 40 wt. %, e.g. 0.1 to 10 wt. %.

In comparison to other gases (nitrogen, carbon dioxide), oxygen (O₂) is associatively bound in very high concentration in emulsions used according to the invention, for example, per kg methylpolysiloxane, up to about 80 g oxygen (O₂) can be bound.

It is advantageous to set a particle size of the silicone oil emulsions in the range of a mean value from 0.3 and 15 μm, e.g. from 2 to 10 μm, or a D99 in the range from 0.5 to 200 μm, e.g. from 5 to 50 μm. The particle sizes here are determined by laser diffractometry with an LS 230 Particle Sizer (Small Volume Module, Beckman-Coulter GmbH, D-Krefeld). The combination of laser diffractometry with the PIDS technology (Polarization Intensity Differential Scattering Technology) enables the measurement of broadly distributed samples with particles in the size range from 40 nm to 2000 μm. The principle of the PIDS technology is the irradiation of the sample to be tested with horizontally and vertically polarized light of different wavelengths. Next, the scattered light intensity at six different angles is analysed (Müller R H, Schuhmann R. Teilchengröβenmessung in der Laborpraxis [Particle size measurement in laboratory practice]: Wissenschaftliche Verlagsgesellschaft mbH Stuttgart; 1996. Chapter 4, 55-99; Keck C M, Müller R H. Size analysis of submicron particles by laser diffractometry-90% of the published measurements are false. Int J Pharm 2008; 355:150-163; Xu R. Improvements in particle size analysis using light scattering. In: Müller R H, Mehnert W. (Ed.) Particle and Surface Characterizing Methods: Scientific Publishers Stuttgart; 1997, Chapter 3, 27-56). The calculation of the particle size distribution from the raw data was performed using the Mie theory and assuming a spherical shape for the particles. For the silicone oil emulsions, a real refractive index for the particles of 1.40 (Wacker Chemie GmbH. Product Information—Wacker Silicone Oils AK. 2006) was used for the calculation. The samples to be characterized were diluted for the measurement with demineralized water. In each case, 6-8 measurements were performed over a period of 90 seconds each, the values from which were then averaged. As measurement parameters of the volume-based distribution, for the assessment of the mean (arithmetic mean), the median (D50 value, central value) and the D99 value (diameter at 99% of total passage) were used.

Emulsions according to the invention are preferably lecithin-free and/or free from hydrophobic silicic acid.

Endogenous diseases can for example be diseases of the nose caused by an immune deficiency, autoimmune reaction or other not clearly definable causes.

Diseases due to exogenous causes can for example be those which are caused by infections, for example by bacteria, fungi or viruses, or allergens, such as dusts or pollen.

Mixed forms with an endogenous and exogenous component are also comprised therein.

“Nasal diseases” or “diseases of the nose” should in particular be understood to mean rhinitis, especially allergic rhinitis (Rhinitis allergica), viral or bacterial rhinitis or that caused by fungi (general infectious) rhinitis, such as for example Rhinitis acuta, or, further, Rhinitis atrophicans (“ozaena”), Rhinitis sicca, hypertrophic rhinitis (Rhinitis hypertrophica), Rhinitis vasomotorica, Rhinitis pseudomembranacea, Rhinitis medicamentosa or chronic rhinitis, or two or more thereof.

Diseases of the nasal mucosa include all (endogenous or exogenous) rhinitis, such as also pollinosis.

Intranasal use is indicated in a mammal, in particular a human, in particular a mammal or human, which/who requires such a treatment.

The production of silicone oil emulsions usable according to the invention is effected by methods known per se, e.g. as described in DE 103 26 188, by homogenization with application of high shear forces after addition of all constituents (optionally also of active ingredients), in particular after addition of the emulsifiers to the aqueous medium in a first step with dissolution or dispersion; or with addition of the emulsifier (e.g. in the case of Span® 80) to the oily phase; and in both cases subsequent combination of the aqueous and the oily phase and pre-homogenization to a pre-emulsion by means of a normal stirrer, a colloid mill, by ultrasonication or by means of a toothed wheel disperser which works on the rotor-stator principle (e.g. Ultra-Turrax® T8, IKA-Werke GmbH & Co KG, D-Staufen) and subsequent high pressure homogenization, e.g. with a high pressure homogenizer, in particular with a ring gap homogenizer, e.g. MicroLab 40 homogenizer (APV-Gaulin, D-Lübeck) or Microfluidizer™ (Microfluidics, USA) by pressing through a narrow ring gap, preferably under a pressure of 100 to 1400 bar and optionally with multiple, e.g. 3- to 10-fold, repetition.

Optionally or if necessary for storage stability, the emulsions are then sterilized, for example autoclaved. Appropriate production processes are described in DE 103 26 188 A1, which is by reference included here.

The enrichment with oxygen is optionally effected as described in EP 2 151 242 A2. The oxygen is physically dissolved, for example by equilibration of the aqueous emulsion of methylpolysiloxane with gaseous oxygen directly before packing, or by aeration of the solution for use after packing or directly before the preparation is taken. For this, appropriately equipped applicators are suitable, for example a gas cartridge with automatic dispensing system built into the drug packaging.

During the use of surface-activated dimethicone which is encapsulated in gas-tight containers, the oxygen binding can already be effected with the oil before filling into the appropriate dosage form. The admixture of 0.5-2% hydrogen peroxide solution or of oxygen-storing compounds such as haem proteins or perfluorocarbons is possible. However, the preferred drug form is the aqueous emulsion of the dimethicones. Here oxygen-saturated dimethicone is used already during the preparation of the emulsion. The first emulsification step is associated with an intensive mixing of a preferably plant emulsifier with the oil phase and the subsequent incorporation into the aqueous phase, in which applications of ultrasound and increases in temperature are also used to improve the emulsification. It should be ensured that the emulsification is performed under an oxygen atmosphere. Thereby, it is ensured that maximal physical binding of oxygen into the dimethicone chains takes place. The content of oxygen should preferably be at least 2%, calculated on the polysiloxane content of the solution. The maximum binding is attained at about 7-8 weight % oxygen based on the oil content. Thus oxygen-containing emulsions preferably have, based on the silicone content, a content of bound oxygen from 1 to 8, e.g. from 6 to 8 or in particular from 7 to 8 wt. %. The oxygen binding in the oily phase is relatively stable and the oxygen is not exchanged with the surrounding emulsion carrier (aqueous medium). The oxygen molecules are specifically enriched associatively bound to the polysiloxane content. The specific binding (without this being intended to be limiting) here is presumably attained in that the alkyl or methyl residues are bound to the respective free Si valences, and form molecular “clamps” for the oxygen molecules since rigidly arranged they possess a weakly negative charge surface onto which the slightly positively charged oxygen molecules can dock or be attached.

The oxygenation can for example be effected with oxygen (e.g. from pressure bottles) in pressure-resistant containers (e.g. to 40 l or more), in which the emulsions are present, preferably under increased pressure, for example from 2 to 10 atm, such as in particular at 2.5 to 5 atm, introduced via an inlet, preferably a frit and with stirring, e.g. for 0.5 to 2 hours, with equilibration for example at room temperature.

A process according to the invention or a method according to the invention for treatment includes in particular the intranasal administration of a nasal formulation or emulsion of silicone oil to be used according to the invention to a mammal, in particular a human, for example as drops or as a nasal spray or by inhaler.

Possible dosages of the dimethicone solutions here lie for example in the range from 0.1 to 5, in particular 0.2 to 2 ml of an (e.g. 1 to 10 wt. %) (in particular dimethicone) emulsion per day, as a rule in at least two portions of 0.1 to 1 ml or with more frequent application, which can contain 0.01 to 5 mg, e.g. 0.1 to 2 mg xylometazoline or oxymetazoline in solution.

Also, the formulations for intranasal use can be provided as emulsions in tubes (similarly to a lotion or as a nasal ointment) or other containers, preferably in the form of nasal drops, for example bottles with pipettes, or in particular as nasal spray for example in spray bottles or other packaging units, or for example as capsules for aerosol-forming (fine atomizing) inhalers.

The silicone oil emulsion as such already enables a treatment of nasal diseases, but it can additionally contain one or more customary active ingredients for the treatment of nasal diseases (=diseases of the nose, in particular of the nasal mucosa).

Here, without this being intended to be a definitive interpretation of the mechanism of action, the action of the silicone oil emulsions without further active substance is possibly attributable to a dispersal (spreading) of the silicone oil on the nasal mucosa with film formation (formation of a thin silicone oil-based protective layer). Together with this or alternatively, directly, for example antimicrobial effects, can also be involved.

If oxygen is additionally contained, a further improved action of the silicone oils, without this being intended to be a definitive interpretation, is possibly attributable to an oxidative action of the oxygen enriched on the nasal mucosa, which on the one hand can react with components of pathogens, and on the other hand could reinforce the defence mechanisms of cells of the nasal mucosa.

An improved action of active ingredients customary for the treatment of nasal diseases becomes possible through the silicone oil emulsions used according to the invention, since firstly the active ingredients can be held longer in direct contact with the nasal mucosa owing to the film formation, and secondly even with reduction of the dosage of the active ingredients compared to the usual nasal remedies containing active ingredients, owing to enrichment of (in particular of lipophilic) active ingredients on the silicone oil droplets of the emulsion, these can be supplied to the nasal mucosa in particularly concentrated form.

Examples of possible active ingredients:

Anti-inflammatory agents such as glucocorticoids, e.g. prednisolone, prednisone, methylprednisone, budesonide, flunisolide, fluocortin butyl, fluticasone 17-propionate, mometasone furoate, betamethasone, hydrocortisone, aminosalicylates, such as sulfasalazine, mesalazine, olsalazine or balsalazide,

Antipyretic agents,

Antibiotics,

Vasodilators,

Enzymes,

Antibodies,

Secretolytic agents,

Mucolytic agents,

α-sympathomimetic agents (in particular phenylephrine, ephedrine, tetryzoline, naphazoline, oxymetozoline, xylometazoline or tramazoline), or indanazoline, etilefrine, fenoxazoline,

antihistamines, such as azelastine, levocabastine, cetirizine, loratidine, terfenadine or fexofenadine;

Leukotriene antagonists, such as prankulast, zafirlukast or montelukast;

β₂-sympathomimetic agents;

Mast cell stabilizers, such as cromoglycic acid or salts thereof, in particular sodium cromoglycate,

Essential oils,

or the like,

pharmaceutically acceptable salts thereof (if salt-forming groups are present), or combinations of two or more of the said active ingredients. Particularly preferred are oxymetazoline and in particular xylometazoline.

Salts are first and foremost usual pharmaceutically usable salts, e.g. (in case of the presence of basic salt-forming groups, such as primary, secondary or tertiary amino groups) acid addition salts; or (in case of the presence of acidic groups, such as —COOH or —SO₃H), salts with bases.

Particularly preferred are α-sympathomimetic agents, in particular one or more of oxymetazoline (quite especially preferred), xylometazoline (quite especially preferred) or naphazoline.

The content by weight of such active ingredients, based on the whole formulation, for example is about 0.01 to 10 wt. %, preferably from 0.05 to 4 wt. %.

The invention also relates to the subjects of the invention described in the claims and the abstract, which are by reference included here.

The following examples serve to illustrate the invention, without limiting its scope:

### EXAMPLE 1

### Production of a Nasal Formulation

Production of an oxygenated silicone oil emulsion: 20 ml silicone oil M 500 MC (Wacker Chemie GmbH) and 500 ml Poloxamer 188 (Lutrol®, BASF, Ludwigshafen, Germany) are added dropwise to 75 ml distilled water in an Ultraturrax® 4× coarsely homogenized at stage 8 for 5 mins and then processed to a ready-for-use stable silicone oil emulsion in a high pressure homogenizer (Yonekura K, Hayakawa K, Kawaguchi M, Kato T. Preparation of stable silicone oil emulsions in the presence of hydroxypropyl methyl cellulose. Langmuir 1998; 14:3145-3148). After storing for one hour and quality control (droplet size, surface activity), the emulsion is introduced into a sterile 100 ml glass bottle with screw closure, which bears a PE plastic capillary (internal diameter 5 mm) of 5 cm length and hermetically sealed. The capillary is attached with a PTE hose to a Conoxia® pressure bottle (Linde AG, Pullach, Germany) with reducing valve and aerated for 60 mins at 4 atm. at room temperature. During the aeration, a small volume of the overlying gas dome is released two to three times via the screw closure, in order to remove from the silicone oil, as far as possible, all foreign gas admixtures possibly that are still present. The oxygenated silicone oil emulsion is then ready for further use.

Production of a stock solution of oxymetazoline or xylometazoline: 500 mg oxymetazoline or xylometazoline (Sigma-Aldrich, Munich, Germany) puriss. are dissolved in 250 ml distilled water and with gentle warming and intensive stirring filled into sterile 25 ml glass bottles and tightly sealed. The bottles are stored at 4° C. until use.

Production of Rhinitis remedy A for the applications below: 5 ml of the oxygenated silicone oil solution and 0.25 ml of the OX/XTZ stock solution are pipetted into a 10 ml nasal application unit (dropper bottle or spray bottle) and made up to volume with 4.75 ml 1.8% NaCl solution. After an intensive shaking mixing phase, the application units are tightly sealed and released for use.

Production of Rhinitis remedy B for the applications below: 100 ml dimethicone SE 2 MC (Wacker Chemie AG, Munich, Germany) are filled into a glass pressure bottle as described above and aerated with oxygen by the method described above. They are processed into a rhinitis remedy analogously to the steps described above.

### EXAMPLE 2

### Use in Patients

By way of example, the formulation from Example 1 was used in patients as follows:

After external cleaning of the nose, the rhinitis remedy is introduced as promptly as possible into each nostril by means of an applicator unit (drop dispenser or pressure spray) (2-3 spray strokes or 3-4 drops) with brief pushing together of the wings of the nose with two fingers. After 10-15 secs the application is complete. If required, this is repeated until the secretion discharge is permanently ended.

Meanings as follows:

SE-2: silicone oil emulsion base=dimethicone SE 2 MC (Wacker Chemie GmbH) (rhinitis remedy B)

XMZ=xylometazoline

OX=oxymetazoline

Data in mg/ml are based on the end concentration of OX or XMZ in the rhinitis remedy.

(A) The following patients were treated with oxygen-saturated SE 2 emulsion with various forms of rhinitis:

Patient 1.

J.O. 39 yrs, female

Diagnosis: acute influenza infection with rhinitis, chronic rhinitis patient with potential dependence:

1^{st} day 4-5 ml/h aqueous-mucous discharge

Application:

1^{st} treatment day: 2 spray strokes SE-2/0.5 mg/ml every 4 h plus 3 spray strokes of OX. before sleep

2^{nd} treatment day: 2 spray strokes every 6-7 h

3^{rd} treatment day: 2 spray strokes every 12 h

4^{th} treatment day: a further 2 spray strokes in the morning

5^{th} day: symptom-free without the viscous purulent discharge otherwise occurring.

Self-assessment from the decade-long use of aqueous nasal sprays with 0.8-1 mg/ml OX:

faster cure with no residues, free paranasal sinuses, no nasal aperture inflammation (“red nose”)—Patient would use the SE-2 emulsion again and again in case of rhinitis.

Patient 2.

J.N. 23 yrs, male

Diagnosis: Rhinitis vasomotorica (allergy to hop protein) massive watery nasal discharge 10-20 mins after ingestion of the allergen (2-3 Tempo handkerchiefs!)

Start of SE 2 emulsion at 0.5 mg/ml plus OX/seawater hourly; within 2-3 h marked decrease in the watery discharge, used 3 times every 12 h, complete remission with no rhinitis-typical mucosal redness and swelling, no tube blockage, no paranasal sinus involvement.

Self-assessment: perfect immediate antagonization of the rhinitis allergy, will use SE-2 NT again immediately, “pleasant drug”.

Patient 3.

P.L. 37 yrs, male

Diagnosis: chronic rhinitis, at least 3 attacks per month, rhinitis mixed form viral-allergic, obligatory use of NT: OX/seawater drugs, nose oils, nose creams;

Treatment for 4 months with SE-2 OX emulsion:

First 2 months: combination with 0.5 mg/ml OX-seawater; for last 2 months SE-2 with 0.25 mg/ml OX-seawater currently in the last month only two subacute rhinitis attacks with 1 spraying every 8 hours, markedly shortened disease episodes: max. 2 days duration.

Self-assessment: very good efficacy with rhinitis attacks becoming ever less frequent, clear superiority over normal commercial OTC products.

Patient 4.

E.N. 71 yrs, female

Diagnosis: severe acute rhinitis with Tracheobronchitis subchronica

1^{st} day: immediately within 7-8 hours severe purulent-watery nasal mucus with paranasal sinus involvement, trigeminal neuralgia and severe left-sided headache, commercial nasal sprays and drops ineffective.

2^{nd} to 4^{th} day: start of application of SE-2/OX saline nasal spray used firstly every 3 h, then after 12 h only twice/day, markedly reduced spray stroke volume and rapid healing of the accompanying inflammatory signs.

Self-assessment: very good efficacy, for the first time healing tendency “marked”.

Patient 5:

H.R. 42 yrs, male

Diagnosis: Rhinitis acuta with viral infection; with at first “blocked nose” due to mucosal swelling on the first day sudden watery nasal discharge with up to ten sneezing attacks per hour.

Start with firstly 3 times each two spray strokes each time of SE-2/0.5 mg/ml XMZ in the first hour, after which remission of the acute secretory phase for more than 4 hours.

Further introduction of SE-2/0.5 mg/ml XMZ, then nasal drops still needed only three times per 16 h, after four days healing without further olfactory disorder.

Self-assessment: good efficacy with secretion of decreased fluid nasal mucus initially still present, which however stopped 4 hours after first spray application.

Patient 6:

C.L. 6 yrs, male

Diagnosis: chronic rhinitis with frequent feverish chill attacks

Immediate use of SE-2/0.125 mg/ml XMZ in seawater with the first symptoms, application (1 spray stroke into each nostril, morning, midday and evening).

Result: no rhinitis, no mucosal redness in the nasal-pharyngeal cavity, no pains.

Assessment by the parents: perfect rhinitis prophylaxis, not yet seen like this with any other rhinitis spray suitable for children, will willingly use again on next infection.

Patient 7:

S.N. 44 yrs, female

Diagnosis: Allergic vasomotor rhinitis on pollen exposure with general symptoms such as headache, subfebrile temperatures and joint problems.

After severe mucosal swelling in the nose and feeling of pressure in paranasal sinuses, start of a purulent mucous rhinitis and “nose pains”, increasing markedly. Use of SE-2/0.25 XMZ several times in the day and 2-3 times in the night because of mouth dryness and inability to breathe through nose.

After 3 days markedly fewer spray applications necessary (twice/day), after 5 days cured.

Self-assessment: rapid diminution in the allergy episodes, otherwise lasting 7-10 days, very good effect.

Patient 8:

C.S. 69 yrs, female.

Diagnosis: Immunodeficiency syndrome with chronic rhinitis involvement, almost permanent catarrh with phases of nasal dryness and mucous nasal discharge.

Use of SE-2/0.25 mg/ml XMZ twice in the day and once in the evening before retiring.

Unambiguous, rapid “healing” of the nasal cavities with no occurrence of rhinitis attacks; application over 14 days, thereafter 4-6 weeks without symptoms.

Self-assessment: very good effect, pleasant “soft” spray quality, will use it again and again.

### EXAMPLE 3

### Comparison of Oxygenated and Non-Oxygenated Dimethicone Emulsion—Antibacterial Action on Larynx Cells

Abbreviations: DIM: dimethicone emulsion
OxiDIM: oxygenated dimethicone emulsion
HEp2 cells human epithelial cell line from the larynx (epithelioma)

In the studies of the efficacy of dilute OxiDIM emulsions with different dimethicone chain lengths on the attachment and the internalization of E. coli on HEp2 cells the non-oxygenated 3% DIM emulsions were also studied. The most striking finding here was that these DIM dilutions without exception even at only 1:3000 still showed a very small inhibition of internalisation.

Experimental procedure: HEp2 cells were incubated to complete cell layer in each of twelve 25 ml cell culture bottles, then during the cell culture medium change, 5000 E. coli-B2332 (from cryoculture, provided by the Institute of Medicinal Microbiology, University Clinic Jena) in 10 ml medium was added in the controls and the five dilutions stages of the 3% dimethicone emulsion (M500) in each case with prior equilibration with Conoxia (O2 (+)) or without aeration (−). After an incubation of 24 hours, the cell layer was rinsed with cell culture medium. Then by means of vital staining, the dead Hep2 cells in 4 quadrants were counted on the inverted microscope. Two tests were performed in the same way with 3% dimethicone emulsion M1000 and M2000.

The results are summarized in the table: on equalization for percentage internalization, a markedly stronger inhibition of internalization can be seen with the oxygenated dimethicone emulsions. The optimal chain length of the dimethicone is to be defined in the range from M 1000-2000.

TABLE
Inhibition of the internalization of E. coli (B2332)
in Hep 2 cells by 3% dimethicone solutions of different
chain lengths (M . . . .) with and without oxygen:

| Oil component | | |
|---|---|---|
| M 500 | M 1000 | M 2000 |

| Oxygen | Yes | No | Yes | No | Yes | No |
|---|---|---|---|---|---|---|
| Control | 180 | 181 | 360 | 385 | 285 | 280 |
| | (100) | (100) | (100) | (100) | (100) | (100) |
| 1:2000 | 128 | 18 | 235 | 315 | 134 | 205 |
| | (71) | (87) | (65) | (88) | (47) | (73) |
| 1:2500 | 132 | 147 | 140 | 240 | 85 | 172 |
| | (73) | (82) | (39) | (67) | (29) | (61) |
| 1:3000 | 105 | 150 | 190 | 225 | 65 | 140 |
| | (58) | (83) | (53) | (80) | (23) | (50) |
| 1:3500 | 95 | 165 | 145 | 285 | 100 | 230 |
| | (53) | (81) | (40) | (80) | (35) | (82) |
| 1:4000 | 190 | 185 | 175 | 290 | 190 | 268 |
| | (106) | (102) | (49) | (81) | (67) | (95) |

Values in brackets: control without oil component=100% in the respective study (M 500, M 1000 or M 2000)

The values at which a stronger inhibition is found with oxygenation than without oxygenation are highlighted by underlining.

The 77% inhibition found with M 2000-OxiDIM for example at the 1:3000 dilution was not to be seen in any experiment with DIM emulsions (Max. inhibition: 54% at 1:2000 dilution 3% M 2000 DIM emulsion).

Result: Non-oxygenated DIM emulsions have a markedly weaker antibacterial effect than OxiDIM emulsions.

### EXAMPLE 4

### Study of Efficacy in Human Patients

The following study design is used in order to confirm the superiority of an oxygen-enriched silicone oil-containing xylometazoline-reduced formulation for nasal application (Group 3) over standard formulation xylometazoline 0.1% clinically and the superiority of oxygen-enriched over non-oxygen-enriched silicone oil-containing formulations:

Design:

Controlled randomized parallel groups study complying with GCP

Groups and Group Size:

4 groups each with 25 participants:
1. Silicone oil
2. Silicone oil plus oxygen
3. Silicone oil plus oxygen plus 0.01% xylometazoline
4. Standard dose xylometazoline 0.1%

Inclusion Criteria/Indication:

Acute rhinitis

Exclusion Criteria:

Drug-induced rhinitis

Severe underlying diseases

Measurement Parameters:

Nasal peak flow

Number of handkerchiefs used

Symptom improvements

Measurement Times:

0,3,7,10 days

Target:

Significant shortening of duration of illness

Significant improvement in the symptoms with non-inferiority of the physical parameters

Safety Parameters:

Full blood count and blood sedimentation at start and end

### EXAMPLE 5

### Use of Oxygenated Silicone Oil Emulsion 2 MC 0.5 Plus “Seawater” Salt Solution 0.5 for Prophylaxis of Acute Viral Rhinitis

Among a group of 5 students who completed a 12 hour intercontinental flight in October 2014, 2 participants (J.N. male 23 years and M.S. male. 24 years) used a spray application of the active substance-free nasal spray (three spray strokes into each nostril each time) before the start, after 4 and after 8 hours. After the end of the flight and in the next few days, the two spray users had no symptoms of acute or allergic rhinitis. The three other participants in the group journey fell ill within 24 hours with a mild influenza infection, subfebrile temperature (37.3-37.8 degrees Celsius) and acute rhinitis. All participants used this nasal spray formulation on the return flight 3 weeks later, and thereafter none of the participants displayed symptoms of acute rhinitis.

1. A method for the intranasal treatment of endogenous diseases of the nose and/or those due to exogenous causes comprising administering a silicone oil-containing formulation.

2. The method according to claim 1, wherein the diseases of the nose are caused by bacteria, fungi, viruses and/or allergens.

3. The method according to claim 1, wherein at least one of the diseases is selected from the group consisting of rhinitis, allergic rhinitis, infectious rhinitis, Rhinitis atrophicans, Rhinitis sicca, Rhinitis hypertrophica, Rhinitis vasomotorica, Rhinitis pseudomembranacea, Rhinitis medicamentosa or chronic rhinitis, and two or more thereof.

4. The method according to claim 1, wherein the silicone oil or the formulation is enriched with oxygen.

5. The method according to claim 1, wherein the formulation comprises an emulsion of silicone oil in an aqueous medium.

6. The method according to claim 1, wherein the formulation comprises an oil-in-water emulsion containing polydialkylsilanes liquid at room temperature in the presence of ionogenic or nonionogenic emulsifiers in an aqueous medium.

7. The method according to claim 6, wherein the polydialkylsilanes have kinematic viscosities in the range from 10 to 5000 mm² s⁻¹ and the emulsifiers are selected from the group consisting of ionogenic emulsifiers, nonionogenic emulsifiers,
Polyoxyethylene sorbitan fatty acid esters,
Mixtures of partial fatty acid esters of sorbitol and anhydrides thereof with oleic acid;
Phospholipids
Block copolymers of ethylene oxide and propylene oxide units;
Hydroxypropylmethylcellulose;
Silicone surfactants in the form of copolymers of polydimethylsilanes and organic glycols or a form thereof obtained by methanol fractionation;
Sucrose esters;
or mixtures of two or more thereof,
or also particulate solids,
in particular of block copolymers of ethylene oxide and propylene oxide units, or further of sucrose units with stearic acid as the predominant acyl content, Polyoxyethylene sorbitan fatty acid esters, alone or mixed with mixtures of partial fatty acid esters of sorbitol and anhydrides thereof with oleic acid, Macrogol glycerol ricinoleates, and phospholipids, and mixtures of two or more thereof.

8. The method according to claim 5, wherein the aqueous medium contains pure water, buffer or salt solutions or seawater, with or without further additives.

9. The method according to claim 1, further containing at least one active ingredient for the treatment of nasal diseases selected from the group consisting of
anti-inflammatory agents, aminosalicylates,
Antipyretic agents,
Antibiotics,
Vasodilators,
Enzymes,
Antibodies,
Secretolytic agents,
Mucolytic agents,
α-sympathomimetic agents, in particular phenylephrine, ephedrine, tetryzoline, naphazoline, oxymetozoline, xylometazoline or tramazoline, or indanazoline, etilefrine, fenoxazoline,
Antihistamines, such as azelastine, levocabastine, cetirizine, loratidine, terfenadine or fexofenadine;
Leukotriene antagonists, such as prankulast, zafirlukast or montelukast;
β₂-sympathomimetic agents;
Mast cell stabilizers, such as cromoglycic acid or salts thereof in particular sodium cromoglycate,
Essential oils,
pharmaceutically acceptable salts thereof, and
combinations of two or more of the said active ingredients.

10. The method according to claim 1, further containing oxymetazoline or in particular xylomethazoline.

11. The method according to claim 1, wherein the formulation is producible with use of a high pressure homogenization step and/or with introduction of oxygen.

12. A method for treating endogenous diseases of the nose and/or those due to exogenous causes, such as bacteria, fungi, viruses or allergens, in particular of the nasal mucosa, in particular rhinitis, allergic rhinitis, infectious rhinitis, further, Rhinitis atrophicans, Rhinitis sicca, Rhinitis hypertrophica, Rhinitis vasomotorica, Rhinitis pseudomembranacea, Rhinitis medicamentosa or chronic rhinitis, or two or more thereof, by administering the formulation of claim 1.

13. A method for treatment of diseases of the nose, in particular the nasal mucosa, comprising the nasal administration of a silicone oil-containing formulation according to claim 1 to a human, in particular which requires such a treatment, in a quantity effective against the said diseases of the nose.

14. The method according to claim 7, wherein the ionogenic emulsifiers comprise sodium dodecylsulphate.

15. The method according to claim 7, wherein the nonionogenic emulsifiers comprise low molecular weight nonionogenic emulsifiers or block copolymers.

16. The method according to claim 15, wherein the low molecular weight nonionogenic emulsifiers or block copolymers comprise Macrogol glycerol ricinoleates.

17. The method of claim 8, wherein the anti-inflammatory agents comprise a glucocorticoid.

18. The method according to claim 17, wherein the glucocorticoid is selected from the group consisting of prednisolone, prednisone, methylprednisone, budesonide, flunisolide, fluocortin butyl, fluticasone 17-propionate, mometasone furoate, betamethasone, and hydrocortisone.

19. The method according to claim 8, wherein the amino salicylates are selected from the group consisting of sulfasalazine, mesalazine, olsalazine, and balsalazide.

20. A composition for treatment of endogenous diseases of the nose and/or those due to exogenous causes comprising a silicone oil-containing formulation.
